SEQUENCE LISTING (24 sequences)

SEQ ID NO: 1 - DNA, 24 bp, Hepatitis C virus
atagccatgggaatgagcacgaat

SEQ ID NO: 2 - DNA, 25 bp, Hepatitis C virus
cgcggatccgtcaggctgaagcggg

SEQ ID NO: 3 - DNA, 24 bp, Hepatitis C virus
atagccatgggataccaagtgcgc

SEQ ID NO: 4 - DNA, 26 bp, Hepatitis C virus
tcccccgggcatcaccccaccatgga

SEQ ID NO: 5 - DNA, 21 bp, Hepatitis C virus
atagccatggaaacccacgtc

SEQ ID NO: 6 - DNA, 25 bp, Hepatitis C virus
cgcggatccgtcatgcgtatgcccg

SEQ ID NO: 7 - DNA, 21 bp, Hepatitis C virus
atagccatggaaacccacgtc

SEQ ID NO: 8 - DNA, 25 bp, Hepatitis C virus
cgcggatccgtcaaatggcccagga

SEQ ID NO: 9 - DNA, 24 bp, Hepatitis C virus
atagccatggcgaagcgctatatc

SEQ ID NO: 10 - DNA, 25 bp, Hepatitis C virus
cgcggatccgtcacagcgacctcca

SEQ ID NO: 11 - DNA, 21 bp, Hepatitis C virus
atagccatggcgcccatcacg

SEQ ID NO: 12 - DNA, 25 bp, Hepatitis C virus
cgcggatccgtcacgtgacaacctc

SEQ ID NO: 13 - DNA, 24 bp, Hepatitis C virus
atagccatggcgagcacctgggtg

SEQ ID NO: 14 - DNA, 25 bp, Hepatitis C virus
cgcggatccgtcagcactcttccat

SEQ ID NO: 15 - DNA, 24 bp, Hepatitis C virus
atagccatggcgtctcagcactta

SEQ ID NO: 16 - DNA, 25 bp, Hepatitis C virus
cgcggatccgtcagcatggagtggt

SEQ ID NO: 17 - DNA, 24 bp, Hepatitis C virus
atagccatgggatccggttcctgg

SEQ ID NO: 18 - DNA, 26 bp, Hepatitis C virus
tcccccgggcatcagcagcacacgac

SEQ ID NO: 19 - DNA, 26 bp, Hepatitis C virus
cgcggatcctgatgtcaatgtcttat

SEQ ID NO: 20 - DNA, 26 bp, Hepatitis C virus
acgcgtcgacgtcatcggttggggag

SEQ ID NO: 21 - DNA, 24 bp, Hepatitis C virus
atagccatgggaatgagcacgaat

SEQ ID NO: 22 - DNA, 28 bp, Hepatitis C virus
cgcggatccgtcacctacgccgggggtc

SEQ ID NO: 23 - DNA, 24 bp, Hepatitis C virus
atagccatgggaatgagcacgaat

SEQ ID NO: 24 - DNA, 28 bp, Hepatitis C virus
cgcggatccgtcagatagagaaagagca

(19) United States
(12) Patent Application Publication
(10) Pub. No.: US 2002/0102534 A1
(43) Pub. Date: Aug. 1, 2002

(54) Exhaustive analysis of viral protein interactions by two-hybrid screens and selection of correctly folded viral interacting polypeptides

(75) Inventors: Pierre LeGrain, Paris (FR); Marc Flajolet, Paris (FR); Giuseppe Rotondo, Selerno (IT); Catherine Transy, Paris (FR); Genevieve Inshauspe, Lyon (FR)

Correspondence Address: FINNEGAN, HENDERSON, FARABOW, GARRETT &, DUNNER LLP, 1300 I STREET, NW, WASHINGTON, DC, 20005, US

(73) Assignee: Institut Pasteur and Institut National de la Sante et de la Recherche Medicale

(21) Appl. No.: 09/962,318

(22) Filed: Sep. 26, 2001

Related U.S. Application Data
(63) Continuation of application No. 09/339,682, filed on Jun. 24, 1999, now abandoned.
(60) Provisional application No. 60/090,894, filed on Jun. 25, 1998.

(51) Int. Cl. C12Q 1/70; C12Q 1/68; G01N 33/53; C12N 7/00

(52) U.S. Cl. 435/5; 435/6; 435/7.1; 435/235.1

(57) ABSTRACT

This invention relates to the detection and analysis of viral protein-protein interactions using a two-hybrid system. This invention allows the definition and use of minimal peptides involved in these protein-protein interactions. In particular, this invention relates to the use of a two-hybrid assay to screen for molecules that interact with hepatitis C virus proteins.

### CROSS-REFERENCE TO RELATED APPLICATIONS

This application relies on, and claims the benefit of, the filing date of U.S. Provisional Application Ser. No. 60/090,894, filed Jun. 25, 1998, the disclosure of which is hereby incorporated herein by reference.

### BACKGROUND OF THE INVENTION

1. Field of the Invention

This invention relates to the detection and analysis of viral protein-protein interactions using a two-hybrid system. This invention allows the definition and use of minimal peptides involved in these protein-protein interactions. In particular, this invention relates to the use of a two-hybrid assay to screen for molecules that interact with hepatitis C virus proteins.

2. Description of Related Art

Most biological processes involve specific protein-protein interactions. General methodologies to identify interacting proteins or to study these interactions have been extensively developed. Among them, the yeast two-hybrid system currently represents the most powerful in vivo approach to screen for polypeptides that could bind to a given target protein. Originally developed by Fields and coworkers (U.S. Pat. Nos. 5,283,173 and 5,468,614, incorporated herein by reference), the two-hybrid system utilizes hybrid genes to detect protein-protein interactions by means of direct activation of a reporter-gene expression (Allen et al., 1995; Transy et al., 1995). In essence, the two putative protein partners are genetically (covalently) fused to the DNA-binding domain of a transcription factor and to a transcriptional activation domain, respectively. A productive interaction between the two proteins of interest will bring the transcriptional activation domain in the proximity of the DNA-binding domain and will directly trigger the transcription of an adjacent reporter gene (usually lacZ or a nutritional marker), giving a screenable phenotype. Transcription can be activated through the use of two functional domains of a transcription factor: a domain that recognizes and binds to a specific site on the DNA and a domain that is necessary for activation, as reported by Keegan et al. (1986) and Ma et al. (1987).

Bartel et al. (1996) extended the approach of the typical two-hybrid system. The approach includes using a known protein that forms a part of a DNA-binding domain hybrid, the hybrid being assayed against a library of all possible proteins present as transcriptional activation domain hybrids, using the genome of bacteriophage T7, such that a second library of all possible proteins fused to the DNA-binding domain to be analyzed. This genome-wide approach to the two-hybrid searches has identified at least 25 interactions among the proteins of T7.

Recently, Rossi et al. (1997) described a different approach, a mammalian “two-hybrid” system, which uses β-galactosidase complementation (Ullmann et al., 1968) to monitor protein-protein interactions in intact eukaryotic cells. Other recent improvements to the two-hybrid assay system are described by Fromont-Racine et al. (1997) and in U.S. patent application Ser. Nos. 09/003,335 and 09/025,151, incorporated herein by reference in their entities.

To date, however, the two-hybrid assay system has not been specifically applied to the systematic study of viral protein-protein interactions other than the bacteriophage T7. As the number of viral genome sequences available increases, there is a great need for new tools directed to the functional and global study of these newly characterized complete or partial genomes.

For example, hepatitis C virus (HCV) is an important etiologic agent of hepatocellular carcinoma (HCC). However, the mechanism of carcinogenesis by HCV is poorly understood. Although liver cirrhosis caused by the virus may be of primary importance in triggering the malignant transformation of hepatocytes, recent evidence suggested that some HCV proteins have transforming capacities and thus can be implicated in the pathogenesis of HCC (Ray et al., 1996; Sakamuro et al., 1995).

The HCV genome is a plus-stranded RNA about 10 kb in length that encodes a single polyprotein of 3009-3010 amino acids processed co- or post-translationally by both cellular and viral proteinases to produce at least 10 mature structural and non-structural viral proteins (FIG. 1). The structural proteins are located in the amino terminal quarter of the polyprotein, and the non-structural (NS) polypeptides in the remainder (for a review see Houghton, 1996). The genome organization resembles that of flavi- and pestiviruses and HCV is now considered to be a member of the Flaviviridae family (Miller and Purcell, 1990; Ohba et al., 1996).

The gene products of HCV are, from the N-terminus to the C-terminus: core (p22), E1 (gp 35), E2 (gp 70), NS2 (p21), NS3 (p70), NS4a (p4), NS4b (p27), NS5a (p58), NS5b (p66). Core, E1, and E2 are the structural proteins of the virus processed by the host signal peptidase(s). The core protein and the genomic RNA constitute the internal viral core and E1 and E2 together with lipid membrane constitute the viral envelope (Dubuisson et al., 1994; Grakoui et al., 1993; Hijikata et al., 1993). The NS proteins are processed by the viral protein NS3 which has two functional domains: one (Cpro-1), encompassing the NS2 region and the N-terminal portion of NS3, which cleaves autocatalytically between NS2 and NS3, and the other (Cpro-2), located solely in the N-terminal portion of NS3, cleaves the other sites downstream NS3 (Bartenschlager et al., 1995; Hijikata et al., 1993).

Due to the lack of a cell culture system supporting efficient HCV replication, efforts to define the HCV-encoded polypeptides have utilized expression of HCV cDNA in cell-free translations and in insect and mammalian cell culture. On the basis of the sequence and genome organization similarities with other members of the Flaviviridae family and recombinant expression, purification and in vitro assay of single virus polypeptide, the function of some HCV proteins have been defined. Immunoprecipitation experiments from extracts of mammalian cells expressing the HCV cDNA have revealed some interactions among virus proteins. The nucleocapsid protein core interacts with one of the envelope glycoprotein, E1, in the membrane of the endoplasmic reticulum (ER) by its C-terminal hydrophobic tail (Lo et al., 1996). An interaction between the two envelope glycoproteins, E1 and E2, has also been detected in the same cellular compartment structure (Dubuisson et al., 1994).

However, the relationship between the virus NS proteins is more difficult to determine using these kinds of experiments. Immunoprecipitation analyses suggest that the NS proteins form a complex. One particular interaction has been well characterized: the interaction between the small hydrophobic protein NS4a and the serine-proteinase domain of NS3 where NS4a acts both as cofactor for the proteinase activity of NS3 on the surface of the ER and as an anchor of the latter in the ER membrane (Bartenschlager et al., 1995; Failla et al., 1995; Kim et al., 1996; Love et al., 1996). Regarding the functions of the NS proteins, the presence of an RNA helicase sequence motif in the C-terminal two-thirds of NS3 and of sequence motifs highly conserved among all the RNA-dependent RNA polymerases (RdRps) within the C-terminal region of NS5b, has led to the prediction of an helicase activity for the C-terminal domain of the former protein and of an RdRp activity for the latter. Both activities have been confirmed in vitro for the two proteins (Behrens et al., 1996; Hong et al., 1996; Suzich et al., 1993). NS5A has been shown to exist in a hyperphosphorylated state (Tanji et al., 1995). However, the function of NS4b and NS5a are not yet known.

One of the characteristics of HCV is its high degree of genetic heterogeneity in vivo, manifested both in the generation of viral quasi-species and in the continuous emergence of neutralization escape mutants (Shimizu et al., 1994). This poses an obstacle to the development of a broadly reactive HCV vaccine based on antibody reactivity to the envelope glycoproteins (Chien et al., 1993). Although alpha interferon has been shown to be useful for delaying the development of HCC in chronically infected HCV patients (Nishiguchi et al., 1995), a highly effective therapeutic agent has not yet been developed to control this important infection and to prevent HCC development. For these reasons, there is a considerable interest in developing HCV-specific antiviral agents that can complement currently available alpha interferon therapy. A detailed understanding of HCV proteins function in connection with virus replication and their interference with the normal cellular genes expression should clarify the mechanisms by which HCV induces hepatocyte transformation and lead to effective means to treat or control the infection. Because HCV does not replicate appreciably in a cell culture-system, impeding efficient basic studies (Jacob et al., 1990; Shimizu et al., 1992), new experimental approaches are needed.

### SUMMARY OF THE INVENTION

This invention provides a method for the detection and analysis of viral protein-protein interactions using a two-hybrid system. In particular, this invention relates to the use of a two-hybrid assay to screen for molecules that interact with hepatitis C virus (HCV) and hepatitis G virus (HGV) proteins.

One of the key issues in the development of efficient therapeutic strategies against viral infection is to understand the network of viral protein:protein interactions necessary for viral replication and propagation. This goal may be reached by building a virus protein linkage map employing a genetic two-hybrid assay on a genome-wide scale. This study of viral protein:protein interactions requires only the availability of the cloned virus genome and its sequence, and overcomes the limitations of other approaches based exclusively on viral protein immunoprecipitation assays. This approach also allows the discovery of new interactions that provide a more detailed understanding and insight into the molecular biology of the virus.

More specifically, the invention provides for the construction of expression systems from a random library derived from a viral genome. These expression systems can be used for the design of multiple biological assays of virus-encoded proteins. Any viral genome or part of a viral genome available as a molecular clone or as a purified nucleic acid sequence can be used for this purpose.

Proper coverage and representation of the initial viral fragment in the constructed library can be insured in at least two ways. Inserts can be prepared by random mechanical or enzymatic cleavage of the cloned viral sequence. Alternatively, PCR primers can be designed from the viral sequence in such a way that their use in combinations allows PCR amplification of sub-fragments spanning the initial sequence. As an example, combinations of sense and antisense primers derived from the HCV coding sequence every 100 nucleotides insures an appropriate coverage of the HCV genome.

The largest viral protein domains revealed by the two-hybrid strategy of this invention will contain several interacting surfaces. These protein domains are then used as baits for the search of host protein partners. Interactions between viral and host proteins can be studied and used in a way similar to those involving only viral products.

The inserts generated by practice of this invention can be cloned by conventional means into any suitable vector appropriate for subsequent applications. Such applications include detection of protein-protein interactions using the two-hybrid system. Using this strategy on an exhaustive basis will reveal all potential protein-protein interactions taking place within the virus. The minimal peptides involved in these interactions can then be defined. The sequences encoding these minimal peptides can in turn be randomly mutated and screened in the same assay for increased affinity for their cognate protein partner. The derived peptides are expected to interfere with the biological functions carried out by the native viral products. The determination of their three-dimensional structure by X-ray diffraction or nuclear magnetic resonance (NMR) will allow the design of structural analogs as anti-viral drugs.

### BRIEF DESCRIPTION OF THE DRAWINGS

FIG. 1 provides a general overview of HCV genome and its encoded polyprotein. The RNA coding strand is represented with a line for untranslated regions (NCR) and boxes for coding regions. Positions and enzymes responsible for cleavage are indicated above. p7 is a secondary cleavage product of E2. Adapted from Houghton, 1996.

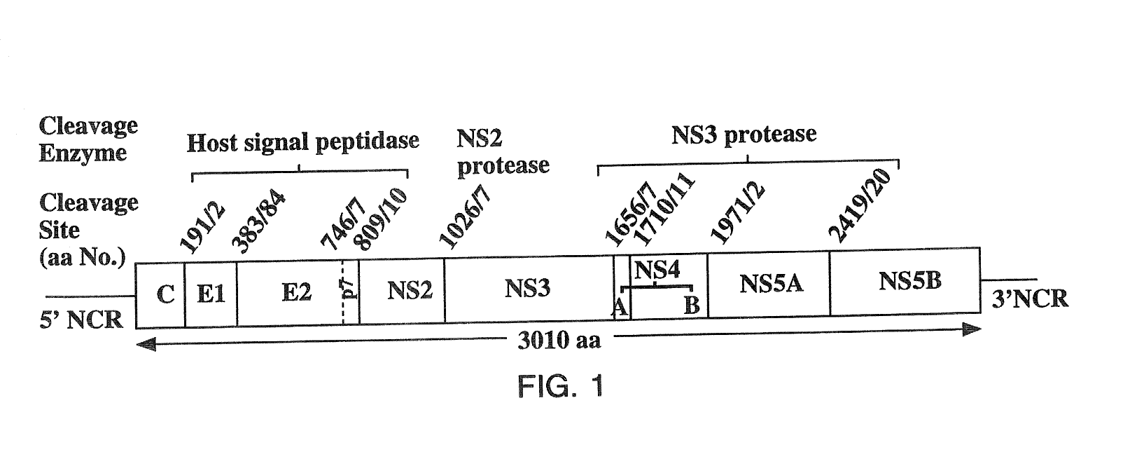

FIG. 2 shows a Western blot analysis of HCV-derived bait proteins. Yeast extracts were prepared from the CG1945 yeast recipient strain, either untransformed (lane 1 and 18) or transformed with bait plasmids (lanes 2 to 17). After separation on polyacrylamide gels and transfer onto membrane, the bait proteins were revealed using a anti-GAL4 (DNA binding domain) monoclonal antibody. The protein fused to the GAL4 DNA binding domain is indicated above each lane. In lane 2, yeast cells expressed only the GAL4 moiety from the pAS2ΔΔ plasmid. Molecular weight markers are indicated in kDa. The bands corresponding to the GAL4 DNA binding domain fusion protein of expected size are indicated by arrowheads.

FIG. 3 provides a matrix analysis of interactions between HCV-derived fusion proteins. The canonical HCV proteins, as well as several truncated versions of these proteins, were cloned into the pAS2ΔΔ plasmid (bait) and into the pACTII plasmid (prey). The three HCV-encoded junctional residues at the N and C termini are indicated. Hydrophobic regions (*) at the N-terminal (NS2) or C-terminal extremities (E1 and E2) of HCV polypeptides were omitted from the constructs. For the E2 protein, two C-terminal extremities were chosen that excluded (E2Δ) or included (E2), part of the p7 fragment (see FIG. 1), according to (Mizushima et al., 1994). For each bait-prey combination, the activity of LacZ and HIS3 reporters is indicated by a square as below the chart. PRP11 and PRP21 are two yeast proteins known to interact with each other and were used as control proteins.

FIG. 4 depicts distribution of prey fragments in the genomic HCV random library. GRBHCV1 E. coli clones were lifted on filters and hybridized with probes covering HCV polypeptide-coding sequences or the complete HCV ORF. Open bars represent calculated distribution and shadowed bars represent the theoretical distribution for polypeptides indicated below.

FIG. 5 depicts a set of preys selected by the CΔ115 capsid bait. A close-up of the HCV genome 5′ end is represented on the top: the 5′ NCR region is indicated by a line and the capsid coding region by a box. The C-terminal boundaries of the three baits used are figured by a vertical bar and the corresponding positions indicated. Only the short CΔ115 bait (filled box) selected preys, indicated below by horizontal lines. The positions of the N-terminal and C-terminal codons of the preys are indicated. Codon 1 corresponds to the initiation codon of the capsid. The number of identical prey clones is indicated into brackets. The junction between untranslated and translated regions is indicated by a dotted line.

FIG. 6 depicts HCV library screening for interaction with HCV-encoded polypeptides. The complete set of preys selected during screens performed with various HCV baits is presented. A schematic view of the coding regions of HCV genome is shown on the top with the positions of codons at the junctions indicated. On the left a similar diagram is shown with the location and size of fragments used as baits. Baits that selected preys are listed on the left and their preys are positioned along the HCV genome. Screens are depicted alternatively in grey or white boxes. Genomic regions in which were found preys selected by the empty bait vector are represented as dark grey boxes.

FIG. 7 provides a detailed analysis of NS3/NS4a interaction using various overlapping fragments. Several combinations of baits (A, B and C) and preys (a to e) were transformed into the yeast strain Y526 (Legrain et al.) and assayed for LacZ activity. The exact position and size of each insert is indicated relative to the NS4a/NS4b/NS5a (baits) and NS2/NS3 (preys) regions, respectively. Experiments were performed on two independent transformants in duplicate. The combinations that were selected during the genomic screens are depicted in boxes. The C construct was subcloned from a prey insert but was not used as bait in a screen.

### DETAILED DESCRIPTION OF PREFERRED EMBODIMENTS

A first aspect of the present invention provides methods for the study and screening of polynucleotides contained in a viral genomic library using a two-hybrid assay system. Preferably, the two-hybrid assays applied to the study of viral genomes follow two principal strategies, which can be combined sequentially for an even more powerful screening method.

The first strategy involves 1) identifying the N-terminus and C-terminus of every known viral protein; 2) cloning the coding sequences into both DNA-binding domain and activation domain vectors; and 3) individually assaying each resulting vector against all of the others in a two-hybrid system to obtain a matrix of viral polypeptide interactions.

The second strategy consists of 1) constructing a library of randomly-generated genomic viral DNA fragments into both DNA binding domain and activation domain vectors; and 2) assaying the library in the DNA-binding vector against the fill library in the activation domain vector by two-hybrid screening.

Both approaches present potential advantages and predictive pitfalls. However, if both strategies are employed independently, and, preferably sequentially or concurrently, they provide confirmatory and complementary information not only about viral protein:protein interactions but also about viral protein folding. For example, in the study of HCV, because the mature HCV proteins are the product of a cis- or trans-processing of the initial polyprotein by the cellular and viral proteinases, their folding follows a precise pathway which may not be reproduced when the DNA coding sequence of each single protein is fused to the DNA binding domain or to the activation domain, as in the above-mentioned first strategy. Mis-folding of the hybrid proteins could prevent the detection of protein interactions. Moreover, with this strategy it is not often possible to define the interacting domains. However, the second strategy provides a much higher probability that, among all HCV fragments fused to both the DNA binding domain and the activation domain represented in the libraries, a subset of protein fragments will fold correctly and the interacting domains will be accessible to each other. This approach also provides data that help to define domains mediating interactions, a necessary step toward the design of inhibitors of such interactions. A problem with this approach is that some of the interactions detected by screening randomly generated libraries may be completely unrelated to a biological protein:protein interaction. That is part of the wider problem of identifying, among positive clones in a two-hybrid screen, those having a biological relevance. However, application of the present invention overcomes many, if not all, of these inherent problems.

In one embodiment of this aspect of the invention, the viral DNA fragments inserted into the library vectors encode less than the full size viral protein for which they are specific. In embodiments, the viral DNA fragments encode between 50% and 75% of the full size of the viral protein. In other embodiments, the viral DNA fragments encode between 30% and 50% of the full size of the viral protein. In other embodiments, the viral DNA fragments encode between 10% and 30% of the full size of the viral protein. In other embodiments, the viral DNA fragments encode between 5% and 10% of the full size of the viral protein.

Any viral genome, or part of a viral genome, that is available as a molecular clone or as a purified nucleic acid sequence can be used in the practice of this invention. Preferably, the viral genome is an HCV or HGV viral genome. The methods of this invention are especially useful for viruses with complex large genomes, such as Herpes viruses, and for viruses in which the folding of the viral proteins is potentially under high constraint, as in the case of HCV. “High constraints” comprises essentially structural constraints, such as those seen in viruses encoding polyprotein precursors, such as flavivirus, and pestivirus groups, which infect humans and animals, and potyviruses, which infect plants.

It is possible to construct the random libraries of this invention in vectors designed for protein expression in a particular type of recipient cells. Such vectors are known in the art. For example, in the case of human recipient cells, vectors maintained as episomes such as those carrying the OriP replication origin of the Epstein-Barr virus, which can be easily rescued from the cells, are especially useful in this application. The viral protein domains can be targeted to the cell compartment appropriate for the subsequent biological assay (e.g., cell surface, secretory pathway, nucleus). Preferred expression vectors are also shuttle vectors.

In a second aspect of this invention, a method of detecting protein:protein interactions is provided. In embodiments of this aspect of the invention, viral protein:viral protein interactions are detected. In other embodiments, viral protein:host protein interactions are detected.

In embodiments, protein:protein interactions taking place within a virus can be identified by utilizing viral genome polynucleotides that encode proteins, or portions thereof, that interact with other viral proteins, polypeptides, or peptides. The terms “peptide”, “polypeptide”, and “protein” refer to polymers in which the monomers are amino acids joined together through amide bonds. Peptides are two or more amino acid monomers long. Polypeptides are more than ten amino acids residues in length. Proteins are more than thirty amino acids residues in length. Thus, “peptides” include polypeptides and proteins, and “polypeptides” include proteins. Standard abbreviations for amino acids are used herein (see Stryer, 1988, Biochemistry, Third Ed., incorporated herein by reference).

In a preferred embodiment, the invention provides a method for detecting viral protein:protein interactions in which the method comprising the steps of:

a) constructing a library of randomly-generated genomic viral DNA fragments in a DNA-binding domain vector;

b) constructing a library of randomly-generated genomic viral DNA fragments in an activation domain vector; and

c) assaying the library in the DNA-binding domain vector with the library in the activation domain vector by two-hybrid screening.

In general, either or both of the libraries can be prepared from a cloned viral genome. For example, the viral genome can be one from a virus such as a herpesvirus, a potyvirus, a flavivirus, and a pestivirus. In highly preferred embodiments, either or both of the libraries is/are prepared from the hepatitis C virus genome or from the hepatitis G virus genome. In embodiments, the cloned viral genome can encode at least one polyprotein precursor. In an embodiment, either or both of said libraries is/are selected from the group consisting of GRBHCVL1 library deposited with the C.N.C.M. under access number I-2039 on Jun. 15, 1998, and GRBHCVL2 library deposited with the C.N.C.M. under the access number I-2040 on Jun. 15, 1998.

In embodiments, protein:protein interactions taking place between viral proteins, polypeptides, or peptides and host cell proteins, polypeptides, or peptides can be identified by utilizing viral genome polynucleotides that encode proteins, or portions thereof, that interact with the host cell proteins, or portions thereof.

For example, a library of the invention can be contacted with hyperimmune serum and resulting immunocomplexes detected. In a preferred embodiment, the method comprises the steps of:

a) contacting expression products from at least one genomic DNA viral library with an hyperimmune serum;

b) visualizing immunocomplexes formed between specific antibodies present in the serum and epitopes present on the expression products; and, optionally,

c) determining the sequence of the expressed epitopes selected.

In preferred embodiments of this aspect of the invention, the interaction of antibodies in the serum with epitopes in the library allows the diagnosis of viral infection. Such a diagnosis can be base on the above method or others according to the invention. For example, diagnosis of viral infection can also be performed by:

a) contacting a biological sample with a library of randomly-generated genomic viral DNA fragments in a DNA-binding domain vector, or in an activation domain vector, under conditions where the viral DNA fragments are expressed; and

b) detecting interaction between expression products from the viral DNA fragments and at least one molecule present in the biological sample;

wherein interaction indicates a viral infection.

It can also be performed by:

a) contacting the biological sample with a collection of from 1 to 100 peptides (including polypeptides and proteins) according to the invention; and

b) detecting interaction between at least one peptide according to the invention with at least one molecule present in the biological sample;

wherein interaction indicates a viral infection.

The random selection strategy of the invention will identify protein fragments constituting structural domains able to fold properly independently of the full-length polypeptide. The minimum peptides (i.e., the smallest functional fragments of the polypeptides) involved in these virus:virus or virus:host interactions can be defined and the information can be used to develop drug screening protocols to identify small molecule inhibitors (e.g., drugs) of those interactions and/or to design and assay peptide inhibitors of such interactions. The sequences of the viral and host cell amino acids and polynucleotides can be determined using techniques

For example, a virus-specific peptide according to the invention, which interacts with a host-encoded protein, can be used in combination with the host protein to screen for molecules that affect the interaction of the peptide with the protein. The molecules can affect the interaction by blocking or reducing it, or they can affect the interaction by facilitating it, such as by increasing the affinity of the peptide for the protein. Alternatively, a viral peptide identified by the present invention can, itself, be used as a therapeutic molecule to, for example, facilitate a biological response. Such a biological response can include, but is not limited to, an immune response, an enzymatic activity, and initiation of a biological cascade.

This invention may also be used to identify viral protein epitopes recognized by immune cells in either HCV-infected patients or healthy individuals. The epitopes can be present on a protein, a polypeptide, or a peptide, and multiple epitopes can be present on each of these molecules. The sorting of all potential epitopes can serve to improve the diagnosis of infection especially during the first stage of the disease. It can also lead to the identification of epitopes eliciting a protective response against infection, and thus be useful for preparing vaccines. In embodiments, the viral protein epitope can be present on a wild-type viral protein. In other embodiments, the viral protein epitope can be a variant of the viral protein epitope, including naturally occurring variants and in vitro mutated variants. “Mutation” or “mutated” as used herein refers to a specific deletion, a specific insertion, or a specific substitution of at least one nucleotide. Thus, a “mutated variant” is a variant that contains a mutation. For example, a mutated triplet codes for a different amino acid than compared to a wild type triplet, and a variant, or mutated variant, can contain this mutated triplet. A variant according to the invention can be specifically made to show altered binding characteristics, with respect to the target protein. That is, the variant can be created, in vitro or in vivo, by known mutagenesis techniques so that it binds to its target with higher or lower affinity. Such variants are useful, for example, in identifying and characterizing drugs which interact with one or both of the proteins.

Another application of the invention is the identification of the viral products that interfere with the host cell metabolism, e.g., the anti-viral host cell defense. For example, several HCV species are known to escape interferon therapy, presumably by inactivating a component of the interferon-induced cell response. Random genomic HCV libraries may be used for the identification of the viral products responsible for the interferon-resistant phenotype. Knowing whether or not this viral product is carried by a particular patient will guide the therapeutic choice.

In another aspect of the invention, libraries are provided which encode proteins capable of interacting with viral proteins, including those which encode a protein, a peptide, and/or a polypeptide. These molecules can be, for example, an antibody, a receptor, a DNA binding protein, a glycoprotein, or a lipoprotein. As used herein, “DNA Binding Protein” refers to a protein that specifically interacts with deoxyribonucleotide strands. A sequence-specific DNA binding protein binds to a specific sequence or family of specific sequences showing a high degree of sequence identity with each other (e.g., at least about 80% sequence identity) with at least 100-fold greater affinity than to unrelated sequences. The dissociation constant of a sequence-specific DNA binding protein to its specific sequence(s) is usually less than about 100 nM, and may be as low as 10 nM, 1 nM, 1 pM, or 1 fM. A nonsequence-specific DNA binding protein binds to a plurality of unrelated DNA sequences with a dissociation constant that varies by less than 100-fold, usually less than tenfold, to the different sequences. The dissociation constant of a nonsequence-specific DNA binding protein to the plurality of sequences is usually less than about 1 μm. In the present invention, DNA binding protein can also refer to an RNA binding protein.

It will be readily apparent to those of skill in the art that application of the methods of this invention will lead to the identification of novel viral polynucleotides and their functions. These polynucleotides and the peptides they encode are within the scope of the invention. The protein, polypeptide, or peptide containing the epitope can be expressed in vitro or in vivo, for instance, using a vector encoding the protein, polypeptide, or peptide. Suitable vectors include retroviral, adenoviral, plasmid, and other vectors for in vitro and in vivo expression. The vector can be administered to an individual and can result in expression of the epitope, providing an immune response against the epitope. According to the invention, the vector for delivering a nucleic acid to a host cell comprises regulatory elements, such as promoter and enhancer, capable of expressing the polynucleotides contained in the vector in human tissue such as muscle, brain, and bone marrow. Such vectors are known in the art.

The identification of viral protein interactions provides pharmaceutical compositions that interfere with the in vivo interaction of viral proteins. “Interfere” as used herein, refers to a positive interference or interaction, which means that the binding is enhanced, or a negative interference or interaction, which means that the binding is decreased or abolished. The methods of the invention also provide epitopes that can elicit a protective response against infection.

Thus, one aspect of the invention is a pharmaceutical composition comprising at least one protein, polypeptide, or peptide, or a polynucleotide molecule (including a vector). The pharmaceutical composition can comprise an acceptable physiological carrier and/or adjuvant, as are known in the art, and can provide a therapeutic effect in those to whom it is administered. The pharmaceutical composition can comprise at least one molecule that interferes with at least one viral protein. It can also comprise at least one molecule that facilitates interaction between two viral proteins, or a viral protein and a host cell protein. In embodiments, it can also comprise a viral peptide, polypeptide, or protein having an epitope against which an immune system generates a response. In embodiments, the pharmaceutical composition can comprise a polynucleotide encoding a protein, polypeptide, or peptide according to the invention. The pharmaceutical composition can be administered by any known route, including, but not limited to, intravenous, intramuscular, subcutaneous, topical, oral, inhalation, and via mucosal surface(s). The therapeutic compositions describe above may be administered to the vertebrate host by a

In a specific embodiment, the invention provides a therapeutic product, comprising a naked polynucleotide operatively coding for a viral peptide according to the invention. The polynucleotide can be in solution in a physiologically acceptable injectable carrier and suitable for introduction interstitially into a tissue to cause cells of the tissue to express the peptide. Therapeutic compositions comprising a polynucleotide are described in the PCT application No. WO90/11092 (Vical Inc.) and also in the PCT application No. WO95/11307 (Institut Pasteur, INSERM, Universite d'Ottawa) as well as in the articles of Tacson et al. (1996, Nature Medicine, 2(8):888-892) and of Huygen et al. (1996, Nature Medicine, 2(8):893-898).

In preferred embodiments, the pharmaceutical composition is an immunogenic composition. The immunogenic composition can comprise, as an immunogenic component, an epitope identified by the methods of the invention. Preferably, the immunogenic response is a protective response. The immunogenic compositions can be used to generate antibodies or to elicit an immunogenic response in an individual into which they are introduced. Antibodies against the epitope can be generated using known techniques, either in humans, for example as part of an immune response, or in animals to obtain large quantities for use in detection of the epitope. Thus, the protein, polypeptide, or peptide according to the invention can be used as part of an immunogenic composition, especially as part of a vaccine.

In an aspect of the invention, a method for delivering a peptide to the interior or a cell of a vertebrate in vivo is provided. This method can comprise the step of introducing a preparation comprising a pharmaceutically acceptable injectable carrier and a naked polynucleotide operatively coding for the polypeptide into the interstitial space of a tissue comprising the cell, whereby the naked polynucleotide is taken up into the interior of the cell and has a pharmaceutical effect. The pharmaceutical effect, in embodiments, is expression, either on the cell surface or as a secreted product, a peptide, polypeptide, or protein, comprising an immunogenic epitope. The epitope is recognized by the host immune system as an antigen, and an immune response is generated against that epitope. Multiple epitopes can also be expressed from one polypeptide, or multiple nucleic acids encoding multiple epitopes can be introduced into the host at the same time.

In an aspect of the invention, a method for delivering a nucleic acid, such as a vector, capable of in vivo expression of a desired amino acid sequence, the vector encoding the desired therapeutic composition as described above is provided. The method comprises administering the vector in a form and an amount sufficient to effect the desired therapy. For example, if the desired effect is to generate an immune response to an encoded epitope, a sufficient amount of vector encoding the epitope is administered to an individual for expression of the epitope in vivo so that the host immune system detects the epitope and generates a response against it. In embodiments, the method comprises administering a vector comprising a polynucleotide according to the invention.

The therapeutic polynucleotide according to the present invention may be injected into the host after it has been coupled with compounds that promote the penetration of the therapeutic polynucleotide within the cell or its transport to the cell nucleus. The resulting conjugates may be encapsulated in polymer microparticles as it is described in the PCT application No. WO94/27238 (Medisorb Technologies International).

In other embodiments, the nucleic acid to be introduced is complexed with DEAE-dextran (Pagano et al. (1967) J. Virol. 1:891) or with nuclear proteins (Kaneda et al. (1989) Science 243:375), with lipids (Felgner et al. (1987) Proc. Natl. Acad. Sci. 84:7413), or encapsulated within liposomes (Fraley et al. (1980) J. Biol. Chem. 255:10431).

The amount of the nucleic acid (e.g., vector) to be injected varies according to the site of injection and also to the kind of disorder to be treated. As an indicative dose, 0, 1, and 100 μg of the vector can be injected in a patient.

In a further aspect of the invention, kits for diagnosis (detection) of viral infections, and kits for therapeutic treatment of viral infections are provided. For example, a diagnostic kit for the detection of a viral infection in a biological sample can comprise at least:

a) a library or a collection;

b) a medium or a support suitable for detecting viral protein:protein interaction and;

c) a medium suitable for revealing the presence of the type of viral protein. A “collection” according to the invention means a group of molecules from a library that has been preliminarily selected.

In embodiments where the kit is designed for therapeutic treatment, therapeutic compositions according to the invention are provided, and the kit can further include ancillary equipment and reagents to be used in administering the compositions, such as antibacterial agents, syringes, sterile diluents, etc.

### EXAMPLES

The following examples serve to illustrate representative embodiments of this invention. The examples are not to be construed as limiting the scope of the invention, but are presented to further clarify specific embodiments of the invention.

### Example 1: Construction of plasmids containing the HCV genome.

Subcloning experiments with the HCV genome were performed using the H strain genome cloned as DNA in a plasmid MINK (pRC/CMV/HCV). This plasmid contains the cDNA genomic sequence of HCV strain H (nt. 1-9416, Inchauspé et al., PNAS, 1991), expressed under the control of the CMV promoter (Invitrogen). The viral sequences correspond to the 5′ untranslated region (5′ UTR), the nucleocapsid, both glycoproteins E1 and E2, the P7 protein, the non-structural proteins NS2, NS3, NS4a and b, NS5a and b, and a truncated 3′ UTR. Briefly, a first clone (named 1968c) was assembled from smaller clones encompassing the 5′ UTR, CAP, E1, E2, NS2 and NS3 (Nt. 1-5398) previously described in Inchauspe et al., 1991 using a PCR-based amplification/ligation approach. The final amplified insert contained a NotI and SspI restriction enzyme sites, respectively, at the 5′ and 3′ end of the sequence, and was cloned into respective sites of the pBluescript II SK-plasmid. Similarly, a second clone was derived (SK-101) after amplification and PCR assembly of HCV sequences encompassing the NS4, NS5a and b and partial 3′ UTR HCV-H sequences (nt. 5377-9416). This clone contains SspI and XbaI sites respectively at the 5′ and 3′ ends of the sequence and was cloned in respective sites of the plasmid pBluescript II SK. After bacterial amplification, both plasmids were digested by the above-indicated restriction enzymes, and inserts were ligated and cloned in corresponding sites from the pBluescript vector to yield clone SK-HCV. The entire HCV insert was further subcloned into the pRC/CMV vector resulting in the pMink vector G28.

### Example 2: Cloning of HCV fragments into expression vectors.

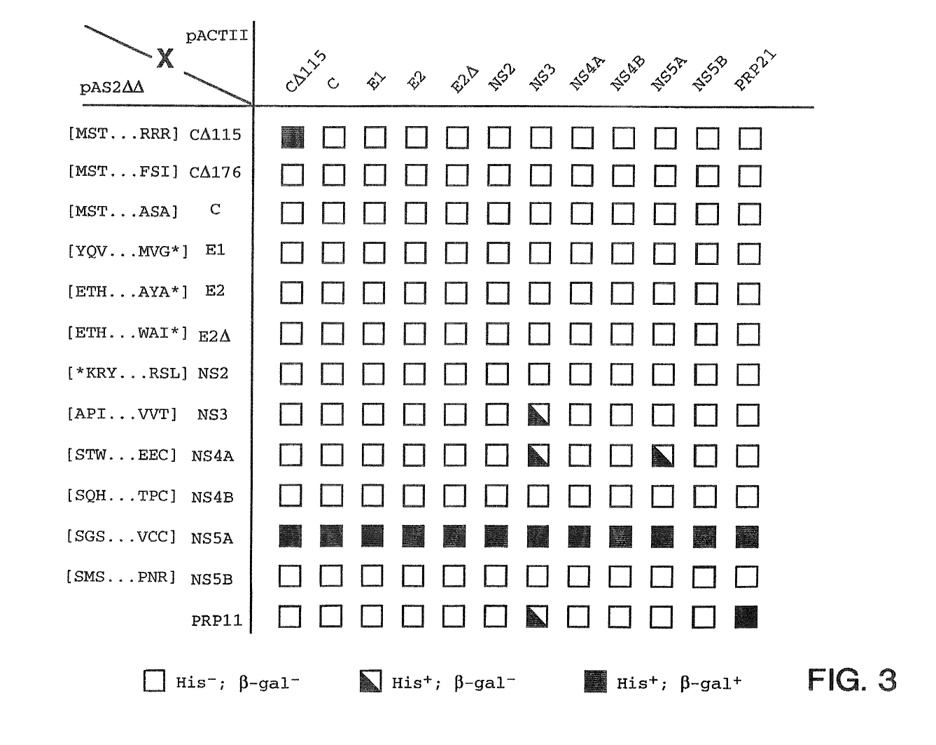

Fragments encoding the canonical HCV polypeptides or derived domains of these proteins as referred in FIG. 3 were obtained by PCR amplification (30 cycles) using primers derived from the cloned HCV genome sequence. The pairs of primers used to amplify the HCV proteins or protein domains are listed below:

| | | |
|---|---|---|
| C | (5′-ATA GCC ATG GGA ATG AGC ACG AAT-3′/5′-CGC GGA TCC GTC AGG CTG | (SEQ ID NO:1) |
| | AAG CGG G-3′) | |
| E1 | (5′-ATA GCC ATG GGA TAC CAA GTG CGC-3′/5′-TCC CCC GGG CAT CAC CCC | (SEQ ID NO:2) |
| | ACC ATG GA-3′) | |
| E2 | (5′-ATA GCC ATG GAA ACC CAC GTC-3′/5′-CGC GGA TCC GTC ATG CGT ATG | (SEQ ID NO:3) |
| | CCC G-3′) | |
| E2D | (5′-ATA GCC ATG GAA ACC CAC GTC-3′/5′-CGC GGA TCC GTC AAA TGG CCC | (SEQ ID NO:4) |
| | AGG A-3′) | |
| NS2 | (5′-ATA GCC ATG GCG AAG CGC TAT ATC-3′/5′-CGC GGA TCC GTC ACA GCG | (SEQ ID NO:5) |
| | ACC TCC A-3′) | |
| NS3 | (5′-ATA GCC ATG GCG CCC ATC ACG-3′/5′-CGC GGA TCC GTC ACG TGA CAA | (SEQ ID NO:6) |
| | CCT C-3′) | |
| NS4a | (5′-ATA GCC ATG GCG AGC ACC TGG GTG-3′/5′-CGC GGA TCC GTC AGC ACT | (SEQ ID NO:7) |
| | CTT CCA T-3′) | |
| NS4b | (5′-ATA GCC ATG GCG TCT CAG CAC TTA-3′/5′-CGC GGA TCC GTC AGC ATG | (SEQ ID NO:8) |
| | GAG TGG T-3′) | |
| NS5a | (5′-ATA GCC ATG GGA TCC GGT TCC TGG-3′/5′-TCC CCC GGG CAT CAG CAG | (SEQ ID NO:9) |
| | CAC ACG AC-3′) | |
| NS5b | (5′-CGC GGA TCC TGA TGT CAA TGT CTT AT-3′/5′-ACG CGT CGA CGT CAT | (SEQ ID NO:10) |
| | CGG TTG GGG AG-3′) | |
| CD115 | (5′-ATA GCC ATG GGA ATG AGC ACG AAT-3′/5′-CGC GGA TCC GTC ACC | (SEQ ID NO:11) |
| | TAC GCC GGG GGT C-3′) | |
| CD176 | (5′-ATA GCC ATG GGA ATG AGC ACG AAT-3′/5′-CGC GGA TCC GTC AGA | (SEQ ID NO:12) |
| | TAG AGA AAG AGC A-3′) | |

For the ease of cloning into the bait vector pAS2ΔΔ, restriction site sequences were added at the 5′ ends of the primers. To minimize the risk of introducing mutations at the PCR step, a DNA polymerase with proof-reading activity (Pfu; Stratagene) was used. In addition, two independent clones of each pAS2ΔΔ construct were analyzed and the junctions between the DBD coding sequence and the HCV insert were determined by nucleotide sequencing. The HCV inserts of the pAS2ΔΔ constructs were recovered by digestion with appropriate restriction enzymes and subcloned into the pACTIIst prey vector. The pACTIIst and pAS2ΔΔ vectors have been previously described by Fromont-Racine et al., 1997, and correspond to prey and bait constructs, respectively. Subcloning from the prey vector to bait vector was performed using cloning sites from polylinkers and following standard procedures.

### Example 3: Western blot analysis of the bait proteins.

Yeast protein extracts were prepared as described by Transy and Legrain, 1995. After separation by SDS PAGE in 10% or 12% gels, the proteins were transferred onto Hybond C extra membranes (Amersham). The membranes were incubated with a monoclonal antibody directed at the GAL4 DNA-binding domain (Santa Cruz) used at a 1:120 dilution and the proteins revealed by chemiluminescence using the Western-star detection kit (Tropix) according to the supplier's instructions.

### Example 4: Matrix analysis of interactions between HCV proteins.

Yeast strains CG1945 and Y187 (Clontech) were used for the two-hybrid screening. Quantitative lacZ reporter assays were made in the Y526 yeast strain. The pAS2ΔΔ-derived plasmids expressing the HCV bait proteins were used to transform the CG1945 yeast strain, a given HCV protein being represented by two independent plasmid clones. One transformant was selected from each transformation plate for re-isolation on -W medium. Similarly the pACTII-derived plasmids expressing the HCV prey proteins were used to transform the Y187 strain and transformants re-isolated on -L plates. The different CG1945 bait transformants were then streaked as patches on a single -W plate to constitute a master plate of the bait matrix. Secondary matrix plates were obtained by replica plating of this master plate. The different Y187 prey transformants were grown at saturation in -L medium. Each of the bait matrices were then replica-plated on one YPGlu plate where an aliquot of a given prey transformant culture had been spread. Cells were allowed to mate by incubation at 30° C. for 16 hours after which replica were performed on -LW plates for the selection of diploid cells. After two days at 30° C., lifts of were prepared onto nylon membranes for lacZ reporter analysis as described by Transy and Legrain, 1995. For HIS3 reporter gene analysis, the different diploid first re-isolated on -LW plates and colonies streaked in parallel on -LW and -LWH growth of colonies was scored after 2 days at 30° C.

### Example 5: Construction of HCV genomic libraries in pACTIIst and pAS2DD vectors.

The base of the library construction strategy have been described by Elledge et al., 1991, and Fromont-Racine et al., 1997. Briefly, 100 μg of recombinant plasmid pMink HCV-H was double-digested with SpeI and XbaI, self-ligated, and sonicated for 15′. DNA was then treated with Mung-bean nuclease, T4 polymerase, and Klenow enzyme. Adapters were prepared as described by Fromont-Racine et al., 1997, and ligated to the sheared HCV-H DNA. DNA was excluded from unligated adaptors on a chroma spin column 200 (Clontech). Forty micrograms of each of pACTIIst and pAS2ΔΔ vectors was digested, dephosphorylated, and partially filled-in. To fill-in the ends of each vector with dGTP, the following reactions were set up:

1) 52 μl pACTIIstop cut BamHI (26 μg) 60 μl Vent polymerase buffer 10x 60 μl dGTP 2mM 415 H2O

2) 57 μl pASAA cut BamHI (20 μg) 30 μl Vent polymerase buffer 10x 30 μl dGTP 172 μl H2O

The reactions were then incubated 5′ at 72° C.

26 units of exo Vent DNA polymerase was added to reaction 1 and 20 units to reaction 2.

The reactions were incubated 1′ at 72° C.

The reactions were then stored on ice until the next step.

The reactions were next extracted with phenol-chloroform and the DNA recovered by ethanol precipitation.

The DNA was dissolved as follows:

pACTIIstop in 50 μl of TE, pH 8 at a concentration of 410 ng/μl, and pASΔΔ in 50 μl of TE, pH 8 at a concentration of 340 ng/μl.

Adaptor-linked HCV DNA was ligated to the pACTIIst and pAS2ΔΔ vectors, respectively, and the E. coli strain MR32 was transformed with each ligation product. Transformant colonies were pooled, aliquots were frozen, and plasmid DNA prepared. These pools constitute the source of genomic HCV fragments cloned into two-hybrid prey (GRBHCVL1 library) and bait (GRBHCVL2 library) vectors, respectively. An aliquot of the GRBHCVL1 library was plated on four 15-cm dishes at a density of 10,000 colonies per plate. Colony lifts onto nylon membranes were hybridized according to standard protocols with [³²P]-labeled probes derived from the different coding regions of the HCV genome. The percentage of colonies containing an HCV insert was estimated by hybridization with a full-length HCV ORF probe.

pACTIIst and pAS2ΔΔ derived libraries were introduced into Y187 and CG1945 yeast cells, respectively. Yeast colonies were pooled and frozen.

### Example 6: Two-Hybrid strategy.

Procedure:

The mating strategy has been previously described by Fromont-Racine et al., 1997. For each screen performed with the HCV/pACTIIst library cloned into the yeast Y187 cells, one vial was thawed and cells were mixed with CG1945 cells transformed with the pAS2DD bait plasmid. Cells were concentrated onto filters and incubated on rich medium for 4½ hours at 30° C. The cells were then collected. A 10⁻³ dilution was spread on -L, -LW, and -W plates to score the number of parental cells and the number of diploids. The rest of the cell suspension was spread on -LWH plates and incubated at 30° C. for three days. After scoring the number of [His ] yeast colonies, 10 ml of an X-Gal mixture (0.5% agar, 0.1% SDS, 6% dimethylformamide and 0.04% X-Gal) were poured on the plates and plates were incubated at 30° C. Blue clones were checked after 30 minutes to 18 hours incubation and streaked on -LWH selective plates. After two-days incubation, an X-Gal lift assay was performed. Double-checked positive colonies were re-streaked. Plasmids were rescued in E. coli, or, alternatively, PCR amplification was performed directly on yeast colonies. Insert junctions with the Gal4 domain were sequenced and precisely identified in the HCV genome.

Few protein-protein interactions detected using full-length HCV polypeptides.

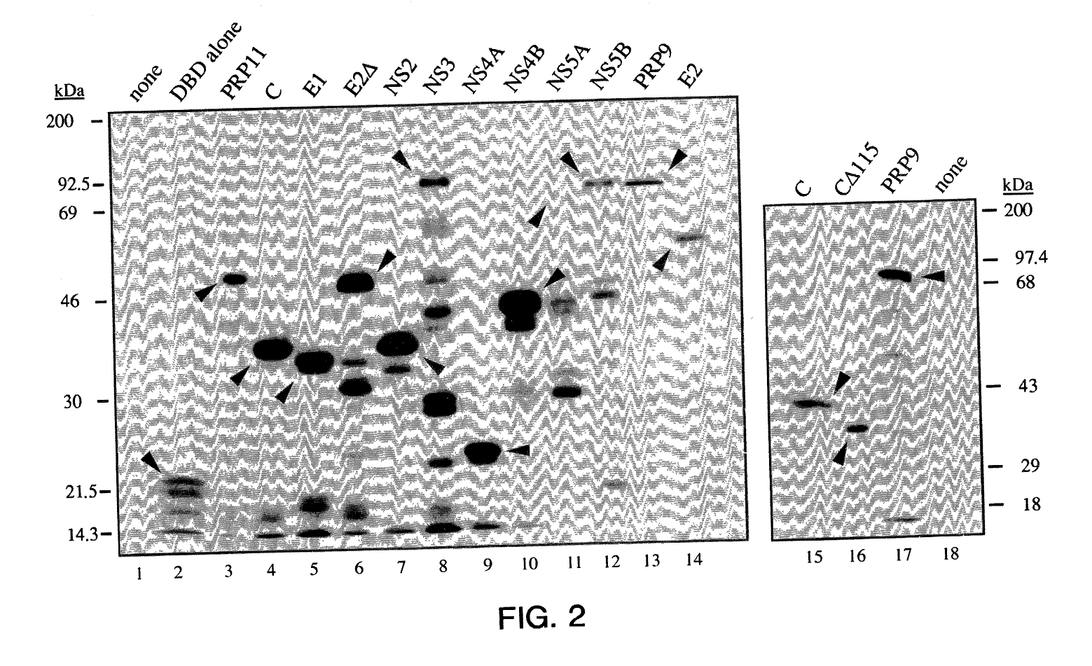

Cleavage products of the HCV polyprotein are well characterized and constitute full length mature HCV proteins. Among those polypeptides, several are supposed or known to interact, such as the capsid that homodimerizes or oligomerizes or the protease NS4a that interacts with the protease domain of NS3. Interactions between all mature HCV polypeptides were assessed in a two-hybrid assay. Production of bait fusion proteins was assayed by Western blot (FIG. 2). All expected products were found expressed, with the notable exception of the NS5a protein being mostly present as a shorter polypeptide than expected. Very few interactions were detected in a two-by-two matrix assay (FIG. 3).

NS5a bait self-activated transcription.

This result has already been reported with truncated mutants of this protein, but not with the full-length protein. The auto-activation that is reported herein could well be due to the processing of the fusion protein (FIG. 2). NS4a weakly interacted with several polypeptides. Surprisingly, the homodimeric interaction of the capsid protein was not detected. In contrast, a truncated version of the capsid protein (Nolandt et al., 1997) interacted with itself but not in combination with the full-length capsid. The interaction of the truncated C protein with other constructs was negative, giving specificity controls for its self-interaction.

Thus, a matrix strategy for the systematic screening of protein-protein interactions yielded poor results. Misfolding or other phenomena probably occur that prevent the use of these chimeric proteins as appropriate tools for protein-protein analyses.

### Example 7: Library against library strategy

Procedure:

Based on the negative results obtained with full length polypeptides fused to Gal4 domains, a screening strategy in which interacting domains could be selected was devised. Due to the small size of most viral genomes, and particularly HCV, it is possible to prepare and screen exhaustive genomic libraries made in both the bait and the prey two-hybrid vectors. However, it may be necessary to screen a high number of different fusion proteins in order to find one that is correctly folded and expressed.

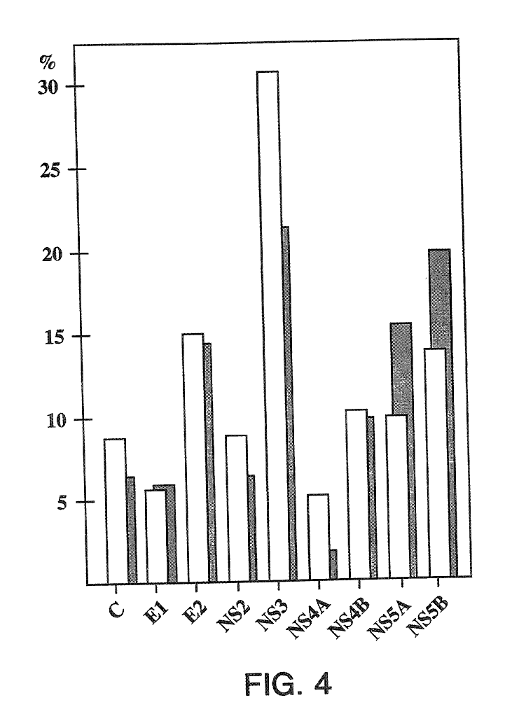

Accordingly, two libraries were made. The first, GRBHCVL1, a prey library, deposited with the National Collection of Cultures of Microorganisms (C.N.C.M.) in Paris under access number 1-2039 on Jun. 15, 1998, contained 40,000 independent pACTIIst derived transformants, fifty per cent of which contained genomic fragments with an average size of 400 bp. The complete HCV genome was well covered as demonstrated by a hybridization experiment performed with the various HCV polypeptides encoding fragments as probes (FIG. 4). Similarly, GRBHCVL2, a bait library, deposited with the C.N.C.M. under access number I-2040 on Jun. 15, 1998, was constructed containing 20,000 independent pAS2ΔΔ derived transformants, eighty per cent of which included a genomic fragment of an average size of 600 bp.

In order to use the powerful mating strategy, the pACTIIst and the pAS2ΔΔ libraries were introduced in the Y187 and CG1945 yeast strains, respectively. 10⁶ bait and 2×10⁵ prey transformant colonies were pooled and aliquots were frozen. Each vial contained several times the original plasmid library. Randomly fused DNA to Gal4 DNA-binding domain often activate transcription of reporter genes on their own. Indeed, replica-plating yeast colonies transformed by pAS2ΔΔ-derived library plasmids led to 10 to 20% auto-activating clones. Two hundred clones, negative for autoactivation, were streaked and used for screens by mating with Y187 yeast cells transformed with the pACTIIst-derived library. 10⁵ potential interactions were assayed in each case. Under these conditions, only 15 baits consistently gave rise to strong His⁺, LacZ⁺ positive colonies when assayed for the prey library screening. Those baits were identified by PCR and sequenced. Only three corresponded to fragments of bona fide HCV polypeptides.

Other baits contained inserts in reverse orientation as to the normal polarity of HCV genome or encoded frameshifted polypeptides as compared to the HCV coding sequence.

These experiments show that randomly picked genetic fragments may act as baits for selecting interacting polypeptides, regardless of the biological meaning of this bait, for example, encoding a polypeptide from the antisense strand of HCV genome. Thus, it appears that the most effective strategy was first to select baits with coding capacity in the HCV genome before performing exhaustive screens.

### Example 8: Screens with full-length polypeptides identify several interactions.

A prey library was screened with predefined baits using protocols adapted from the yeast genome screening (Fromont-Racine et al., 1997). Theoretically, a 95% coverage of the HCV initial prey library of 4×10⁴ clones in E. coli is achieved with 12×10⁴ transformed yeast colonies. Therefore, the screening by mating strategy required three times more yeast diploid cells, i.e., roughly 5×10⁵ clones. This number was reached for most screens (Table 1), suggesting that the set of identified partners reflected a large coverage of the library.

Table 1. Characteristics of HCV library screens

Genomic screens were performed with various polypeptides as baits. For each screen, the number of interactions tested is indicated as the number of diploid cells obtained in the mating experiment. Colonies that grew on selective medium for the HIS3 reporter were counted and subjected to a Lac Z assay. Most of the Lac Z⁺colonies were further characterized by sequencing the corresponding genomic insertion.

| Baits | Number of diploids (10⁵) | His⁺ | LacZ⁺ | identified |
|---|---|---|---|---|
| pAS2DD | 8 | 400 | 39 | 28 |
| Core | 14 | 3 | 0 | — |
| CoreD115 | 63 | 41 | 26 | 16 |
| E1 | 4.4 | 2 | 0 | — |
| E2 | 6.4 | 8 | 4 | 4 |
| E2D | 2.4 | 0 | 0 | — |
| NS2 | 25 | 0 | 0 | — |
| NS3 | 5.6 | 55 | 6 | 3 |
| NS4a | 2.4 | 166 | 14 | 13 |
| NS4b | 16 | 20 | 0 | — |
| NS5a | 38 | autoact. | — | — |
| NS5b | 3.2 | 17 | 0 | — |
| pGR1 | 5.6 | 60 | 0 | — |
| pGR2 | 10 | 1527 | 80 | 45 |
| pGR3 | 12 | 349 | 28 | 16 |
| pGR4 | 12 | 14 | 0 | — |
| pGR5 | 1.6 | 0 | 0 | — |
| pGR6 | 35 | 210 | 143 | 83 |
| pGR7 | 2 | 10 | 0 | — |
| pGR8 | 6.4 | 75 | 10 | 6 |
| pGR9 | 8 | 193 | 26 | 18 |
| pGR10 | 15 | 3 | 5 | 3 |
| pGR12 | 70 | 1260 | 87 | 65 |
| pGR13 | 17 | 896 | 57 | 57 |

The library was first screened with the empty pAS2ΔΔ vector. His+, LacZ⁺positive clones were sequenced. Most of them mapped within three regions of the genome. This result demonstrates first that selection indeed operated and that the screen was saturated since identical fragments were selected several times. Second, it identified HCV genomic regions in which preys activate a transcription of reporter genes without interaction with a HCV encoded bait polypeptide. Many selected fusions in the E2 protein start in a very narrow range of nucleotides located in the endoplasmic domain of E2 some of them being out of frame. They may represent an interaction with an artifactual polypeptide or, alternatively, lead to the production of a HCV encoding polypeptide via a frameshifting event (Fromont-Racine et al., 1997). There are two out of frame fusions starting close to each other at the beginning of the NS3 helicase domain. Finally, two independent fusions were found in NS5b. Since these three HCV regions were selected with the Gal4 DNA binding domain alone, they were not considered as significant and specific preys when found in screens with other baits.

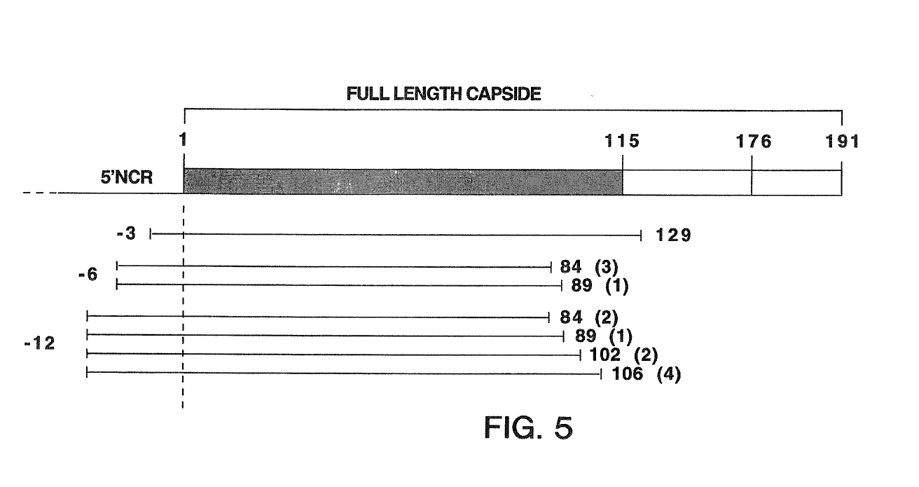

Exhaustive screens were then made with all full length HCV proteins as baits. The numbers of selected preys in these screens are given in Table 1. As expected auto-activation with NS5a was observed. For the other proteins, only E2, NS3, and NS4a baits selected His⁺, LacZ⁺colonies. Unexpectedly, no partner was selected with the core protein. The truncated core fusion protein coreal Δ115 was also used in a screen and selected highly positive colonies. The results are striking (FIG. 5). 14 out of 16 sequenced preys fell in the core sequence. The selection of multiple independent overlapping fragments allows definition of a minimal fragment encompassing the homodimerization domain. The initiation codon is essential (all selected fragments were fused upstream of this codon), and there was clearly a limitation for homodimerization with fragments encompassing amino acid 130 (the only selected clone that contains residues downstream of position 107 was only weakly positive). This is in agreement with the finding that full length core polypeptides do not homodimerize in a two hybrid assay (Nolandt et al., 1997).

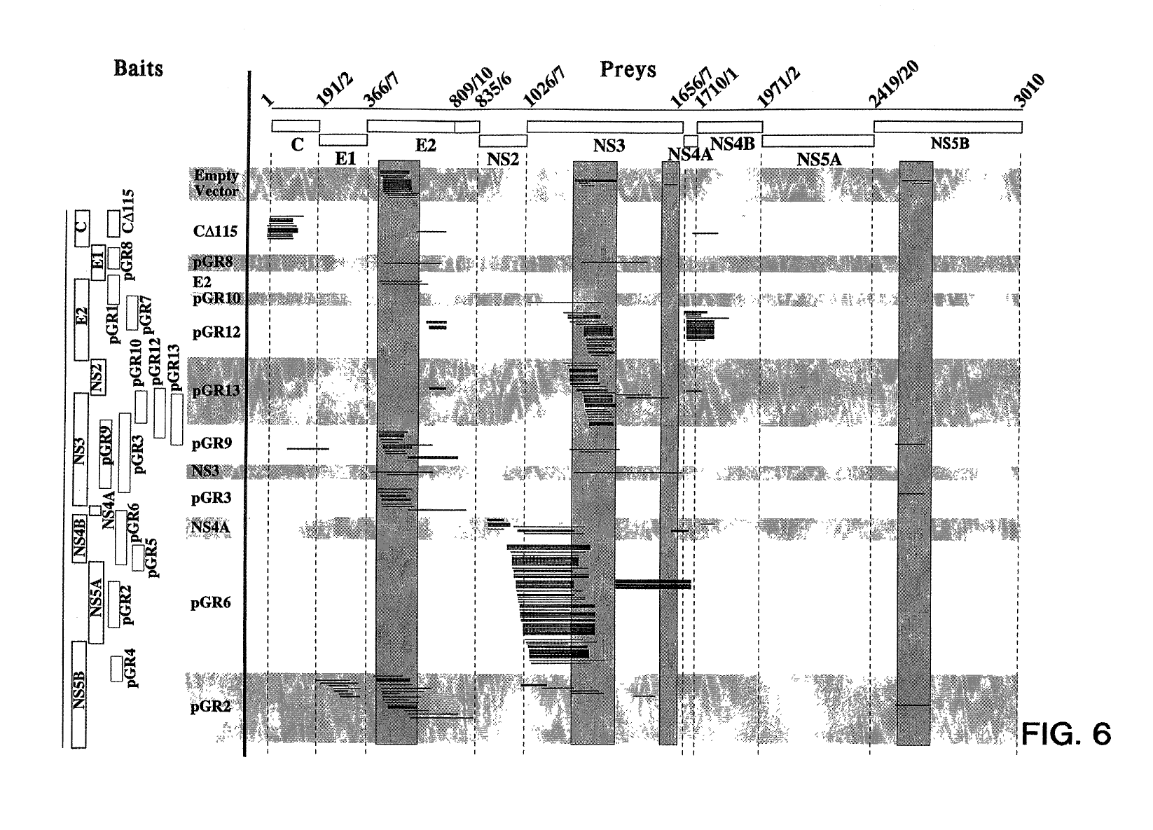

Selected fragments in the various screens were identified and compared to the preys selected against the empty vector (FIG. 6). E2 and NS3 proteins selected only preys also found in the pAS2ΔΔ vector screen. In the NS4a screen, two groups of overlapping fragments were selected as preys, one spanning a central region of NS2 and the other, the protease domain of NS3. In addition, two additional preys were found, one spanning the COOH-terminus of NS3 and the NH2 terminus of NS4a and another fusion spanning part of NS4b.

### Example 9: Screens with randomly selected fragments identify novel interactions.

Randomly located baits were selected by sequencing randomly picked pAS2ΔΔ derived plasmids. Those found in the positive orientation and in frame were assayed by Western blot for production of the fusion protein and for absence of autoactivation (pGR1 to pGR10). Screens were performed (Table 1) and again preys were selected only in a few cases. Preys are indicated in FIG. 6. pGR3, 8, and 9 selected preys that fell within the regions selected by the empty vector. On the contrary, pGR2 and pGR6 selected specific preys. These baits were located in the NS5a and the NS4a/b-NS5a, respectively. The former one selected specific clones within E1 while the latter selects mostly preys within the NS2-NS3 region. Several preys selected in various screens fell in the C-terminal part of E2 (FIG. 6). Those partners are considered as non-specific since they were selected with various independent baits.

In order to further characterize the NS4a/NS3 interaction as precisely as possible, two of the preys located in the protease domain of NS3 were in turn cloned as baits and the prey library was screened (pGR12 and pGR13, Table 1). They share a large fragment and are fused one hundred nucleotides from each other. pGR12 spans the NS2/NS3 boundary, whereas pGR13 is completely included in the NS3 protein. Screens performed with pGR12 and pGR13 selected specific and non-specific preys (FIG. 6). Within the former category, NS4a overlapping fragments were selected although much more often with pGR12 than with pGR13 bait.

### Example 10: Interactions identified between HCV polypeptides are specific.

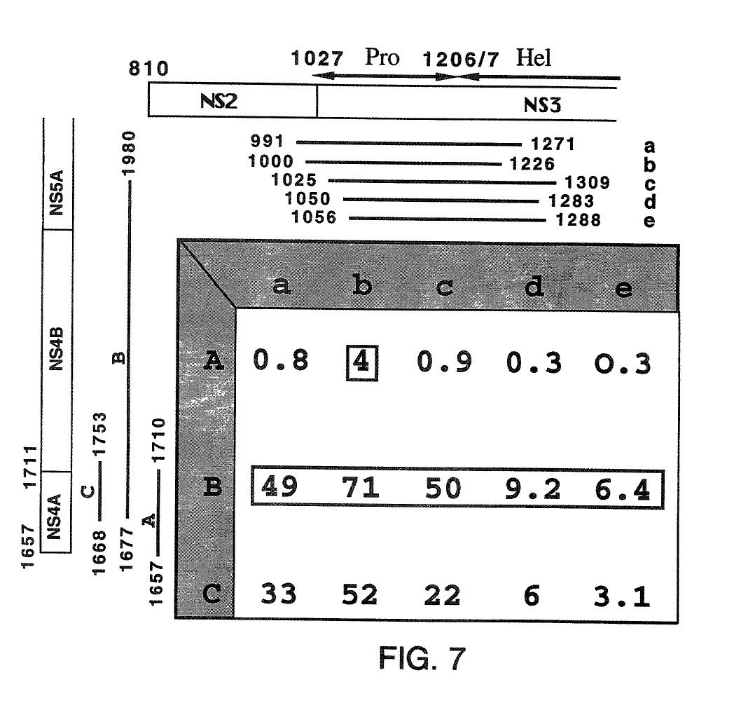

To verify the specificity of selected interactions between HCV encoded polypeptides, a matrix experiment was performed in which selected preys were tested against various HCV-encoded bait polypeptides. As a whole this experiment confirmed the interactions found in the screens. In other words, i) NS3 interacts with NS4a, using various constructs overlapping these polypeptides; ii) NS4a interacted with NS2 although this interaction was not detected using NS2 fragment as a bait and NS4a as a prey; and iii) NS4a interacted with NS4b. Thus, specific interactions were selected in two-hybrid screens of the HCV genome. This was further demonstrated by analyzing more precisely the well characterized NS3/NS4a interaction. Many overlapping fragments were selected in those regions allowing a measurement of the LacZ reporter activity for various combinations of baits and preys (FIG. 7). NS4a full length protein is not an efficient bait whereas its C-terminal moiety is sufficient to interact with NS3 overlapping fragments. The fusion of this region with the complete NS4b protein up to the N-terminal region of NS5a (original pGR6 bait, FIG. 6) does not change the efficiency of interaction. Similarly, the N-terminal region of the NS3 protein is required for efficient binding to NS4a since fusions that do not encompass the starting residue of NS3 do not interact strongly with NS4a (fusions d and e compared to a, b or c). These results are in agreement with the published results that state that NS3 fragment starting at residue 1049 is not an efficient protease and does not bind to NS4a (Satoh et al., 1995).

References:

Behrens, S. E., Tomei, L., and De Francesco, R. (1996), “Identification and properties of the RNA-dependent RNA polymerase of hepatitis C virus”, Embo J. 15:12-22.

Chen, P.-J., and Chen, D.-S. (1997), “Hepatitis B virus and hepatocellular carcinoma”, Liver Cancer, K. Okuda and E. Tabor, eds.

Chien, D. Y., Choo, Q. L., Ralston, R., Spaete, R., Tong, M., Houghton, M., and Kuo, G. (1993), “Persistence of HCV despite antibodies to both putative envelope glycoproteins”, Lancet 342:933.

Dubuisson, J., Hsu, H. H., Cheung, R. C., Greenberg, H. B., Russell, D. G., and Rice, C. M. (1994), “Formation and intracellular localization of hepatitis C virus envelope glycoprotein complexes expressed by recombinant vaccinia and Sindbis viruses”, J. Virol. 68:6147-60.

Elledge, S. J., Mulligan, J. T., Ramer, S. W., Spottswood, M., and Davis, R. W. (1991), “Lambda YES: a multifunctional cDNA expression vector for the isolation of genes by complementation of yeast and Escherichia coli mutations”, Proc. Natl. Acad. Sci. USA 88:1731-1735.

Failla, C., Tomei, L., and De Francesco, R. (1995), “An amino-terminal domain of the hepatitis C virus NS3 protease is essential for interaction with NS4A”, J. Virol. 69:1769-77.

Fields, S., and Song, 0. (1989), “A novel genetic system to detect protein-protein interactions”, Nature 340:245-246.

Fromont-Racine, M., Rain, J. C., and Legrain, P. (1997), “Toward a functional analysis of the yeast genome through exhaustive two-hybrid screens”, Nat. Genet. 16:277-282.

Grakoui, A., Wychowski, C., Lin, C., Feinstone, S. M., and Rice, C. M. (1993), “Expression and identification of hepatitis C virus polyprotein cleavage products”, J. Virol. 67:1385-95.

Hijikata, M., Mizushima, H., Akagi, T., Mori, S., Kakiuchi, N., Kato, N., Tanaka, T., Kimura, K., and Shimotohno, K. (1993), “Two distinct proteinase activities required for the processing of a putative nonstructural precursor protein of hepatitis C virus”, J. Virol. 67:4665-75.

Hijikata, M., Mizushima, H., Tanji, Y., Komoda, Y., Hirowatari, Y., Akagi, T., Kato, N., Kimura, K., and Shimotohno, K. (1993), “Proteolytic processing and membrane association of putative nonstructural proteins of hepatitis C virus”, Proc. Natl. Acad. Sci. USA 90:10773-7.

Hong, Z., Ferrari, E., Wright-Minogue, J., Chase, R., Risano, C., Seeling, G., Lee, C.-G., and Kwong, A. (1996), “Enzymatic Characterization of Hepatitis C Virus NS3/4A Complexes Expressed in Mammalian Cells by Using the Herpes Simplex Virus Amplicon System”, J. Virol. 70:4261-68.

Houghton, M. (1996). Hepatitis C virus, Fields, ed.

Inchauspéet al., “Genomic structure of the human prototype strain H of hepatitis C virus, Comparison with American and Japanese Isolates”, Proc. Natl. Acad. Sci. USA 88:1092-10296.

Jacob, J. R., Burk, K. H., Eichberg, J. W., Dreesman, G. R., and Lanford, R. E. (1990), “Expression of infectious viral particles by primary chimpanzee hepatocytes isolated during the acute phase of non-A, non-B hepatitis”, J. Infect. Dis. 161:1121-7.

Kim, J. L., Morgenstern, K. A., Lin, C., Fox, T., Dwyer, M. D., Landro, J. A., Chambers, S. P., Markland, W., Lepre, C. A., O'Malley, E. T., Harbeson, S. L., Rice, C. M., Murcko, M. A., Caron, P. R., and Thomson, J. A. (1996), “Crystal structure of the hepatitis C virus NS3 protease domain complexed with a synthetic NS4A cofactor peptide”, Cell 87:343-55.

Kolykhalov et al. (Science, 1997, 277, 570-574).

Kuo, G., Choo, Q. L., Alter, H. J., Gitnick, G. L., Redeker, A. G., Purcell, R. H., Miyamura, T., Dienstag, J. L., Alter, M. J., Stevens, C. E., and et al. (1989), “An assay for circulating antibodies to a major etiologic virus of human non-A, non-B hepatitis”, Science 244:362-4.

Legrain et al., “Interactions between PRP9 and SPP91 splicing factors identify a protein complex required in prespliceosome assembly”, Genes and Development, 7:1390-1399 (1993).

Lo, S.-Y., Selby, M., and OU, J.-H. (1996), “Interaction between Hepatitis C Virus Core Protein and E1 Envelope Protein”, J. Virol. 70: 5177-82.

Love, R. A., Parge, H. E., Wickersham, J. A., Hostomsky, Z., Habuka, N., Moomaw, E. W., Adachi, T., and Hostomska, Z. (1996), “The crystal structure of hepatitis C virus NS3 proteinase reveals a trypsin-like fold and a structural zinc binding site”, Cell 87:331-42.

Miller, R. H., and Purcell, R. H. (1990), “Hepatitis C virus shares amino acid sequence similarity with pestiviruses and flaviviruses as well as members of two plant virus supergroups”, Proc. Natl. Acad. Sci. USA 87:2057-61.

Mizushima, H., Hijikata, M., Asabe, S.-I., Hirota, M., Kimura, K., and Shimotohno, K. (1994), “Two hepatitis C virus glycoprotein E2 products with different C termini”, J. Virol. 68:6215-6222.

Nishiguchi, S., Kuroki, T., Nakatani, S., Morimoto, H., Takeda, T., Nakajima, S., Shiomi, S., Seki, S., Kobayashi, K., and Otani, S. (1995), “Randomized trial of effects of interferon-alpha on incidence of hepatocellular carcinoma in chronic active hepatitis C with cirrhosis”, Lancet 346:1051-5.

Nolandt, O., Kern, V., Muller, H., Pfaff, E., Theilmann, L., Welker, R., and Krausslich, H. G. (1997), “Analysis of hepatitis C virus core protein interaction domains”, J. of General Virology 78:1331-40.

Ohba, K., Mizokami, M., Lau, J. Y., Orito, E., Ikeo, K., and Gojobori, T. (1996), “Evolutionary relationship of hepatitis C, pesti-, flavi-, plantviruses, and newly discovered GB hepatitis agents”, FEBS Lett 378:232-4.

Okuda, K. (1997), “Hepatitis C virus and hepatocellular carcinoma”, Liver Cancer, K. Okuda and E. Tabor, eds., pp. 39-50.

Ray, R. B., Lagging, L. M., Meyer, K., and Ray, R. (1996), “Hepatitis C virus core protein cooperates with ras and transforms primary rat embryo fibroblasts to tumorigenic phenotype”, J. Virol. 70:4438-43.

Sakamuro, D., Furukawa, T., and Takegami, T. (1995), “Hepatitis C virus nonstructural protein NS3 transforms NIH 3T3 cells”, J. Virol. 69:3893-6.

Satoh, S., Tanji, Y., Hijikata, M., Kimura, K., and Shimotohno, K. (1995), “The N-terminal region of hepatitis C virus nonstructural protein (NS3) is essential for stable complex formation with NS4A”, J. Virol. 69:4255-4260.

Shimizu, Y. K., Hijikata, M., Iwamoto, A., Alter, H. J., Purcell, R. H., and Yoshikura, H. (1994), “Neutralizing antibodies against hepatitis C virus and the emergence of neutralization escape mutant viruses”, J. Virol. 68:1494-500.

Shimizu, Y. K., Iwamoto, A., Hijikata, M., Purcell, R. H., and Yoshikura, H. (1992), “Evidence for in vitro replication of hepatitis C virus genome in a human T-cell line”, Proc. Natl. Acad. Sci. USA 89:5477-81.

Suzich, J. A., Tamura, J. K., Palmer-Hill, F., Warrener, P., Grakoui, A., Rice, C. M., Feinstone, S. M., and Collett, M. S. (1993), “Hepatitis C virus NS3 protein polynucleotide-stimulated nucleoside triphosphatase and comparison with the related pestivirus and flavivirus enzymes”, J. Virol. 67:6152-8.

Tanji, Y., Kaneko, T., Satoh, S., and Shimotohno, K. (1995), “Phosphorylation of hepatitis C virus-encoded nonstructural protein NS5A”, J. Virol. 69:3980-3986.

Transy, C., and Legrain, P. (1995), “The two-hybrid: an in vivo protein-protein interaction assay”, Mol. Biol. Rep. 21:119-127.

Yanagi et al. (PNAS, 1997, 94, 8738-8743).

All references cited herein are hereby incorporated in their entirety by reference.

What is claimed is:

1. A method for detecting viral protein:protein interactions, said method comprising the steps of:
a) constructing a library of randomly-generated genomic viral DNA fragments in a DNA-binding domain vector;
b) constructing a library of randomly-generated genomic viral DNA fragments in an activation domain vector; and
c) assaying the library in the DNA-binding domain vector with the library in the activation domain vector by two-hybrid screening.

2. The method of claim 1, wherein either or both of said libraries is prepared from the hepatitis C virus genome or from the hepatitis G virus genome.

3. The method of claim 1, wherein either or both of said libraries is prepared from a cloned viral genome that is from a virus selected from the group consisting of herpes virus, potyvirus, flavivirus, and pestivirus.

4. The method of claim 1, wherein either or both of said libraries is prepared from a cloned viral genome that encodes a polyprotein precursor.

5. The method of claim 1, wherein either or both of said libraries is selected from the group consisting of GRBHCVL1 library deposited with the C.N.C.M. under access number I-2039 on Jun. 15, 1998, and GRBHCVL2 library deposited with the C.N.C.M. under the access number 1-2040 on Jun. 15, 1998.

6. A method for detecting viral protein:protein interactions, said method comprising the steps of:
a) constructing a library of DNA fragments in a DNA binding domain vector, wherein at least one DNA fragment encodes at least one molecule that interacts with viral proteins, and wherein said at least one molecule is selected from the group consisting of protein, polypeptide, and peptide;
b) constructing a library of DNA fragments in an activation domain vector, wherein at least one DNA fragment encodes at least one molecule that interacts with viral proteins, and wherein said at least one molecule is selected from the group consisting of protein, polypeptide, and peptide; and
c) assaying the library in the DNA-binding vector with the library in the activation domain vector by two-hybrid screening.

7. The method of claim 6, wherein said protein is selected from the group consisting of an antibody, a receptor, a DNA binding protein, a glycoprotein, and a lipoprotein.

8. The method according to claim 1, wherein at least one peptide is expressed from the library in the DNA-binding vector and wherein the peptide is a variant molecule compared to the known wild type viral peptide.

9. The method according to claim 8, wherein the variant peptide presents at least one mutation selected from the group consisting of deletion, substitution, and insertion of at least one amino acid residue.

10. A peptide detected by the method of claim 1.

11. A pharmaceutical composition comprising at least one molecule that interferes with at least one viral protein, said at least one molecule that interferes being detected by the method of claim 1.

12. The pharmaceutical composition of claim 11, further comprising an acceptable physiological carrier and/or adjuvant.

13. The pharmaceutical composition of claim 12, wherein said composition is administered by a route selected from the group consisting of an intravenous route, an intramuscular route, an oral route, and a mucosal route.

14. A method for detecting specific viral protein epitopes in a biological sample, said method comprising the steps of:
a) contacting expression products from at least one of said libraries of claim I with an hyperimmune serum;
b) visualizing immunocomplexes formed between specific antibodies present in said serum and epitopes present on said expression products; and, optionally,
c) determining the sequence of the expressed epitopes selected.

15. An immunogenic composition comprising at least one epitope that elicits a protective response against infection, wherein said at least one epitope is detected by the method of claim 14.

16. A peptide detected by the method of claim 14.

17. A therapeutic composition comprising at least one peptide according to claim 16.

18. A method for delivering an in vivo expression vector encoding the peptide of claim 16, said method comprising administering said vector to an individual.

19. A method of diagnosing a viral infection in a biological sample, said method comprising the steps of:
a) contacting the biological sample with a library of randomly-generated genomic viral DNA fragments in a DNA-binding domain vector, or in an activation domain vector, under conditions where said viral DNA fragments are expressed; and
b) detecting interaction between expression products from said viral DNA fragments and at least one molecule present in said biological sample;
wherein interaction indicates a viral infection.

20. A method of diagnosing a viral infection in a biological sample, said method comprising the steps of:
a) contacting the biological sample with a collection of from 1 to 100 peptides according to claim 10; and
b) detecting interaction between at least one polypeptide according to claim 10 with at least one molecule present in said biological sample;
wherein interaction indicates a viral infection.

21. A diagnostic kit for the detection of a viral infection in a biological sample, said kit comprising at least:
a) a library or a collection;
b) a medium or a support suitable for detecting viral protein:protein interaction and;
c) a medium suitable for revealing the presence of the type of viral protein.
